(19) United States
(12) Patent Application Publication
(10) Pub. No.: US 2022/0411592 A1
(43) Pub. Date: Dec. 29, 2022

(54) METHOD FOR INDUCING GELATION AND BIOMIMETIC MINERALIZATION OF SILK FIBROIN SOLUTION BY ALKALINE PHOSPHATASE

(71) Applicant: SOOCHOW UNIVERSITY, Suzhou (CN)

(72) Inventors: Xinming LI, Suzhou (CN); Hang LI, Suzhou (CN)

(21) Appl. No.: 17/598,051

(22) PCT Filed: Aug. 20, 2020
(86) PCT No.: PCT/CN2020/110116

§ 371 Date: Aug. 5, 2022

(30) Foreign Application Priority Data
Jul. 29, 2020 (CN) 202010745099.3

(51) Int. Cl. C08J 3/075; A61L 27/36; A61L 27/52; A61L 27/22; C12N 9/18

(52) U.S. Cl. CPC C08J 3/075; A61L 27/3604; A61L 27/52; A61L 27/227; C12N 9/18; A61L 2430/02; C12Y 301/03001

(57) ABSTRACT

The invention provides a method for inducing gelation and biomimetic mineralization of a silk fibroin solution by alkaline phosphatase. A micromolecular polypeptide that is sensitive to ALP and has good biocompatibility and self-assembly property is introduced as a gelator precursor, which can remove a phosphate group under the catalytic action of ALP to generate NY, to trigger supramolecular self-assembly, and therefore SF co-self-assembly is synergistically induced, finally resulting in rapid formation of SF hydrogel. ALP wrapped in an SF-NY hydrogel network still retains its catalytic activity and catalyzes beta-glycerophosphate to release free phosphate ions, so that formation of apatite minerals is induced in the gel. The biomimetic mineralized SF gel can be used as a biomimetic scaffold to promote the adhesion, proliferation and osteogenic differentiation of rat bone marrow mesenchymal stem cells in vitro, and can also promote the natural healing of femoral defects in a rat model.

This application is the National Stage Application of PCT/CN2020/110116, filed on Aug. 20, 2020, which claims priority to Chinese Patent Application No. 202010745099.3, filed on Jul. 29, 2020, which is incorporated by reference for all purposes as if fully set forth herein.

### FIELD OF THE INVENTION

The present invention relates to the technical field of materials, and more particularly to a method for inducing gelation and biomimetic mineralization of a silk fibroin solution by alkaline phosphatase.

### DESCRIPTION OF THE RELATED ART

In nature, the biomineralization is affected by many organic components, including proteins, polysaccharides and enzymes, and these organic components play an important role in regulating the growth of hydroxyapatite crystals. In the process of natural bone formation, alkaline phosphatase (ALP) secreted by osteoblasts releases inorganic phosphate ions from organic phosphates, thereby increasing the local phosphate concentration and promoting hydroxyapatite (HA) mineralization. In the water environment, alkaline phosphatase can catalyze the removal of a phosphate group on a substrate molecule, making the substrate more hydrophobic. In 2004, Xu et al. reported for the first time that under the catalysis of alkaline phosphatase, a substrate molecule Fmoc-pY (Fmoc=fluorenylmethoxycarbonyl, pY=phosphotyrosine) was removed of a phosphate group to generate Fmoc-Y, a hydrogel was formed under the π-π interaction, and also, a nanofiber network structure was formed by self-assembly, where the storage modulus of the hydrogel was about 1000 Pa. Then, starting from this pioneering work, a large number of peptide hydrogels constructed based on phosphatase catalysis, including Fmoc-FpY, Ac-YYYpY-OMe (Ac=acyl), Nap-GFFpY-OMe (Nap=naphthyl), Nap-FFGEpY, NapFFpY, etc., have been reported successively.

Silk fibroin is a natural polymeric fibrin, in which glycine (Gly), alanine (Ala) and serine (Ser) are more than 80% of the fibrin. Because of its excellent biocompatibility, controllable biodegradability, and good flexibility and tensile strength, silk fibroin has been extensively studied by scientists. A large number of biological materials, such as nanofibers, sponges, films, microspheres, hydrogels, etc., that are constructed with silk fibroin as a base material have been reported successively, and are widely used in the repair of various body tissues, such as bone tissue, skin, blood vessels, nerves, tendons and ligaments. Among them, silk fibroin hydrogel is favored by researchers because of its fiber structure similar to natural extracellular matrix, high water content, adjustable porosity, and good affinity with cells. However, the gelation process of a silk fibroin solution is very slow under physiological conditions. For example, at room temperature, it takes over 14 days for a silk fibroin solution with a concentration of 2.0% to transform from a solution to a gel under physiological conditions. Therefore, generally, gelation takes place only under acidic conditions (pH=about 4) or higher temperatures (60° C.). All such factors greatly limit the wide application of silk fibroin hydrogel in the field of biomedicine. In order to change the characteristics of low pH, high temperature and long time required for silk fibroin gelation, scientists have done a lot of research. For example, physical methods such as ultrasonic treatment, vortex shearing, and electrifying are used to induce the secondary structure of silk fibroin to transform from a random-coil conformation in solution to a β-sheet conformation in the gel state, thereby accelerating the gelation process of silk fibroin. Scientists also add chemical reagents such as organic reagents, inorganic composites, ionic liquids, high-pressure carbon dioxide, surfactants, and synthetic polymers to a silk fibroin solution to adjust the interaction with the silk fibroin chain, thereby changing the gel property of silk fibroin and promoting the rapid forming of silk fibroin gel. In addition, scientists also use poly(ethylene glycol diglycidyl ether) (PGDE), 1-(3-dimethylaminopropyl)-3-ethylcarbodiimide hydrochloride (EDC), genipin, chloroauric acid, etc. as a chemical crosslinking agent to prepare silk fibroin gel materials with good mechanical strength and stability.

However, these traditional methods still face some challenges and shortcomings when they are applied in clinical medicine. For example, when the silk fibroin solution is rapidly gelled by physical methods such as ultrasonic treatment, vortex shearing, and electrification, the gelation process under non-physiological conditions that is triggered by electronic instruments does not match the clinical medical environment. By adding chemical reagents such as organic reagents, inorganic composites, ionic liquids, high-pressure carbon dioxide, surfactants and synthetic polymers into the silk fibroin solution, although the gelation time of silk fibroin is shortened to a certain extent, this series of the gelation processes are incompatible with certain clinical environments, and have shortcomings such as potential cytotoxicity of organic molecules, biological inertness of high-molecular polymers, and difficulty of degradation in the body. In addition, although scientists also use poly(ethylene glycol diglycidyl ether) (PGDE), 1-(3-dimethylaminopropyl)-3-ethylcarbodiimide hydrochloride (EDC), genipin, chloroauric acid, etc. as a chemical crosslinking agent to obtain silk fibroin gel materials with good mechanical strength and stability, the potential cytotoxicity of the residual chemical cross-linking agent in the system affects the biocompatibility of the silk fibroin gel materials. In summary, although these measures can shorten the silk fibroin gelation time to a certain extent, the resulting silk fibroin gel materials have poor biocompatibility and greater cytotoxicity. These problems have caused their application in biomedical materials to be greatly restricted.

### SUMMARY OF THE INVENTION

In order to solve the above problems, the present invention induces the gelation and biomimetic mineralization of silk fibroin (SF) through continuous catalytic reaction triggered by alkaline phosphatase (ALP). In this system, a micromolecular polypeptide (NYp), which is sensitive to ALP and has good biocompatibility and excellent self-assembly property, is introduced as a gelator precursor; the gelator precursor can be removed of a phosphate group on the molecule under the catalytic action of ALP to generate NY, supramolecular self-assembly is triggered, and therefore SF co-self-assembly is synergistically induced, finally resulting in rapid formation of SF hydrogel. ALP wrapped in an SF-NY hydrogel network still retains its catalytic activity and catalyzes beta-glycerophosphate to release free phosphate ions, so that formation of apatite minerals is induced in the gel.

A first object of the present invention is to provide a method for inducing gelation of a silk fibroin solution by alkaline phosphatase, comprising the following steps: adding a self-assembling micromolecular polypeptide in a silk fibroin solution as a gelator precursor, to obtain a mixed solution of the silk fibroin solution and the self-assembling micromolecular polypeptide, and adding alkaline phosphatase into the mixed solution, to remove a phosphate group on the molecule of the self-assembling micromolecular polypeptide by the alkaline phosphatase, to trigger supramolecular self-assembly and induce silk fibroin co-self-assembly, forming a silk fibroin gel material.

Preferably, the self-assembling micromolecular polypeptide is selected from 2-naphthalene acetic acid-glycine-phenylalanine-phenylalanine-phosphotyrosine (NYp), 2-naphthalene acetic acid-phenylalanine-phenylalanine-lysine-phosphotyrosine (NapFFKYp), 2-naphthalene acetic acid-phenylalanine-phenylalanine-phosphotyrosine (NapFFYp) and any combination thereof.

Preferably, the concentration of the silk fibroin in the mixed solution is 0.1%-2.0%.

Preferably, the concentration of the self-assembling micromolecular polypeptide in the mixed solution is 0.05 wt %-0.3 wt %.

Preferably, the amount of the alkaline phosphatase added is 10 U/mL-40 U/mL.

Preferably, the pH of the mixed solution is 7-8.

A second object of the present invention is to provide a silk fibroin gel material prepared by the method.

A third object of the present invention is to provide a method for biomimetic mineralization of the silk fibroin gel material, comprising the following steps: adding the silk fibroin gel material into a mineralizing solution and culturing for 5-10 days to obtain a biomimetic mineralized hydrogel, the mineralizing solution comprising 10-40 mM CaCl₂ and 6-20 mM β-glycerophosphate (β-GP).

A fourth object of the present invention is to provide a biomimetic mineralized hydrogel prepared by the method.

A fifth object of the present invention is to provide use of the biomimetic mineralized hydrogel in the preparation of body tissue repair materials.

The present invention has the following advantageous effects:

The present invention induces the gelation and biomimetic mineralization of silk fibroin (SF) through continuous catalytic reaction triggered by alkaline phosphatase (ALP). In this system, the inventors introduce a micromolecular polypeptide (NYp) that is sensitive to ALP and has good biocompatibility and excellent self-assembly property as a gelator precursor; the gelator precursor can be removed of a phosphate group on the molecule under the catalytic action of ALP to generate NY, supramolecular self-assembly is triggered, and therefore SF co-self-assembly is synergistically induced, finally resulting in rapid formation of SF hydrogel. ALP wrapped in an SF-NY hydrogel network still retains its catalytic activity and catalyzes beta-glycerophosphate to release free phosphate ions, so that formation of apatite minerals is induced in the gel. Due to the mild gelation process and the formation of apatite minerals in the gel matrix, the biomimetic mineralized SF gel can be used as a biomimetic scaffold to promote the adhesion, proliferation and osteogenic differentiation of rat bone marrow mesenchymal stem cells (rBMSCs) in vitro, and can also promote the natural healing process of femoral defects in a rat model.

### BRIEF DESCRIPTION OF THE DRAWINGS

FIG. 1 shows solid-phase synthesis steps of a polypeptide molecule NYp;

FIG. 2 shows (a) a gelator precursor NYp solution (0.08 wt %, pH=7.4); (b) a supramolecular hydrogel formed by the supramolecular self-assembly of the NYp solution catalyzed by ALP (10 U/mL); (c) strain sweep and (d) frequency sweep in dynamic rheological test of the NY supramolecular hydrogel (NY=0.08 wt %, pH=7.4, ALP=10 U/mL);

FIG. 3 shows (a) an SF solution with a concentration of 2.0%; (b) an SF hydrogel with a concentration of 2.0%; (c) strain sweep and (d) frequency sweep in dynamic rheological test of the SF hydrogel (SF=2.0%, pH=7.4, ALP=10 U/mL);

FIG. 4 shows the gelation process and mechanical property of a hybrid gel Gel 1; (a) a NYp solution (0.16 wt %, pH=7.4); (b) an SF solution (0.2%, pH=7.4); (c) a hybrid gel Gel 1 containing NY (0.08 wt %) and SF (0.1%) at pH=7.4 and ALP=10 U/mL; (d) strain sweep and (e) frequency sweep in dynamic rheological test of the Gel 1 hydrogel;

FIG. 5 shows the gelation process and mechanical property of a hybrid gel Gel 2; (a) a NYp solution (0.2 wt %, pH=7.4); (b) an SF solution (0.2%, pH=7.4); (c) a hybrid gel Gel 2 containing NY (0.1 wt %) and SF (0.1%) at pH=7.4 and ALP=10 U/mL; (d) strain sweep and (e) frequency sweep in dynamic rheological test of the Gel 2 hydrogel;

FIG. 6 shows the gelation process and mechanical property of a hybrid gel Gel 3; (a) a NYp solution (0.4 wt %, pH=7.4); (b) an SF solution (0.2%, pH=7.4); (c) a hybrid gel Gel 3 containing NY (0.2 wt %) and SF (0.1%) at pH=7.4 and ALP=10 U/mL; (d) strain sweep and (e) frequency sweep in dynamic rheological test of the Gel 3 hydrogel;

FIG. 7 shows the gelation process and mechanical property of a hybrid gel Gel 4; (a) a NYp solution (0.6 wt %, pH=7.4); (b) an SF solution (0.2%, pH=7.4); (c) a hybrid gel Gel 4 containing NY (0.3 wt %) and SF (0.1%) at pH=7.4 and ALP=10 U/mL; (d) strain sweep and (e) frequency sweep in dynamic rheological test of the Gel 4 hydrogel;

FIG. 8 shows the gelation process and mechanical property of a hybrid gel Gel 5; (a) a NYp solution (0.6 wt %, pH=7.4); (b) an SF solution (1.0%, pH=7.4); (c) a hybrid gel Gel 5 containing NY (0.3 wt %) and SF (0.5%) at pH=7.4 and ALP=10 U/mL; (d) strain sweep and (e) frequency sweep in dynamic rheological test of the Gel 5 hydrogel;

FIG. 9 shows the gelation process and mechanical property of a hybrid gel Gel 6; (a) a NYp solution (0.6 wt %, pH=7.4); (b) an SF solution (2.0%, pH=7.4); (c) a hybrid gel Gel 6 containing NY (0.3 wt %) and SF (1.0%) at pH=7.4 and ALP=10 U/mL; (d) strain sweep and (e) frequency sweep in dynamic rheological test of the Gel 6 hydrogel;

FIG. 10 shows the gelation process and mechanical property of a hybrid gel Gel 7; (a) a NYp solution (0.6 wt %, pH=7.4); (b) an SF solution (4.0%, pH=7.4); (c) a hybrid gel Gel 7 containing NY (0.3 wt %) and SF (2.0%) at pH=7.4 and ALP=10 U/mL; (d) strain sweep and (e) frequency sweep in dynamic rheological test of the Gel 7 hydrogel;

FIG. 11 shows the gelation process and mechanical property of a hybrid gel Gel 8; (a) a NYp solution (0.6 wt %, pH=7.4); (b) an SF solution (4.0%, pH=7.4); (c) a hybrid gel Gel 8 containing NY (0.3 wt %) and SF (2.0%) at pH=7.4 and ALP=20 U/mL; (d) strain sweep and (e) frequency sweep in dynamic rheological test of the Gel 8 hydrogel;

FIG. 12 shows the gelation process and mechanical property of a hybrid gel Gel 9; (a) a NYp solution (0.6 wt %, pH=7.4); (b) an SF solution (4.0%, pH=7.4); (c) a hybrid gel Gel 9 containing NY (0.3 wt %) and SF (2.0%) at pH=7.4 and ALP=40 U/mL; (d) strain sweep and (e) frequency sweep in dynamic rheological test of the Gel 9 hydrogel;

FIG. 13 shows the scanning electron microscope (SEM) images and energy dispersive spectroscopy (EDS) data of the biomimetic mineralizated hydrogel materials at different calcium ion (Ca²⁺) concentrations; (a) and (d) the calcium ion concentration is 10 mM; (b) and (e) the calcium ion concentration is 20 mM; (c) and (f) the calcium ion concentration is 50 mM. (g) X-ray diffraction analysis, (h) Fourier transform infrared spectroscopy analysis and (i) X-ray photoelectron spectroscopy analysis of HA and SF-NY gel (SF=2.0%, NY=0.3 wt %, ALP=10 U/mL) and Ca-20 gel (SF=2.0%, NY=0.3 wt %, ALP=10 U/mL, Ca²+=20 mM) hydrogels before and after biomimetic mineralization.

FIG. 14 shows (a) dead and live stained fluorescence images and (b) corresponding cell density statistics after rat bone marrow mesenchymal stem cells (rBMSCs) are cultured on the surface of a blank culture plate, SF-NY gel and Ca-20 gel for 1, 4, and 7 days; (c) cytotoxicity test of SF-NY gel and Ca-20 gel (CCK8 method);

FIG. 15 shows qRT-PCR detection of expression of osteogenesis-related genes and proteins (a) Runx2, (b) Col 1α, (c) OCN, and (d) OPN;

FIG. 16 shows (a) two-dimensional Micro-CT images and (b) three-dimensional reconstructed Micro-CT images at 4 and 8 weeks after femoral surgery in rats; (c) quantitative analysis results at 4 and 8 weeks after femoral surgery in rats: bone mineral density (BMD), bone volume to total tissue volume ratio (BV/TV), trabecular thickness (Tb.Th), and trabecular space (Tb.Sp).

### DETAILED DESCRIPTION OF THE PREFERRED EMBODIMENTS

The present invention will be further described below in conjunction with drawings and specific examples, so that those skilled in the art can better understand and implement the present invention, but the examples described are not intended to limit the present invention.

2-Chlorotrityl chloride resin (100 to 200 mesh, 0.3 to 0.8 mmol/g), Fmoc-Tyr(H₂PO₃)—OH, Fmoc-Gly-OH, Fmoc-Phe-OH and HBTU (benzotriazole-N,N,N′,N′-tetramethyluronium hexafluorophosphate) were purchased from GL Biochem (Shanghai) Ltd; DIEA (N,N-diisopropylethylamine) was purchased from Energy Company; 2-naphthaleneactic acid was purchased from Sinopharm; other organic solvents were ordered from Jiangsu Qiangsheng Company.

### Example 1: Preparation and Purification of a Silk Fibroin Solution

(1) Silk Degumming

4.24 g of anhydrous sodium carbonate was weighted and dissolved in 2 L of boiling deionized water (Na₂CO₃ concentration: 0.02 M), and 5.0 g of silk was added and boiled for 1 h. During the boiling, the silk should be peeled off frequently to avoid entanglement into bundles. Then, the silk was removed and scrubbed with deionized water for 3-4 times, and dried in air overnight at room temperature. The degummed silk was weighted to be 3.5 g, which accounts for about 70% of the total weight of the silk.

(2) Silk Dissolution

32.3 g of anhydrous LiBr was weighed and formulated into 40 mL of a 9.3 M solution, and filtered with filter paper. 2 g of the degummed silk was weighed and added into 12 mL of the LiBr solution, and heated at 60° C. with slow stirring for 4 h.

(3) Solution Dialysis

First, a dialysis bag was washed with deionized water for 2-3 times; then, the silk fibroin solution dissolved in the LiBr solution was added into the dialysis bag, and the dialysis bag was placed into the deionized water environment for dialysis, where ionized water was changed every other hour and the dialysis lasted for at least 72 h.

(4) Solution Concentration

After the dialysis was completed, the dialysis bag was removed and placed into a crystallizing dish with a diameter of 20 cm. PEG 20000 was applied to the surface of the dialysis bag to absorb water, and could be replenished after PEG 20000 on the surface of the dialysis bag was substantially dissolved, until the silk fibroin solution became slightly yellow.

(5) Solution Centrifugation

The concentrated silk fibroin solution was transferred to a 50 mL centrifuge tube and centrifuged twice at 4° C. and 9000 r/min for 20 min each time, and the supernatant was collected.

(6) Concentration Determination

The mass of a clean petri dish was weighed and recorded as m₀; 1 mL of the centrifuged silk fibroin solution was pipetted into the petri dish, which was weighed and recorded as m₁. The petri dish containing the silk fibroin solution was placed into an oven at 60° C. overnight, removed, cooled to room temperature, weighed and recorded as m₂. 5 duplicate samples were taken and the concentration average was calculated. The concentration calculation formula was as follows:

$$C = \frac{{m}_{2} - {m}_{0}}{{m}_{1} - {m}_{0}} \times 100 \%$$

TABLE 1

| | m₀/g | m₁/g | m₂/g | (m₁ − m₀)/g | (m₂ − m₀)/g | c |
|---|---|---|---|---|---|---|
| A | 3.4135 | 4.2114 | 3.4766 | 0.7979 | 0.0631 | 7.9% |
| B | 4.2625 | 5.0429 | 4.3240 | 0.7804 | 0.0615 | 7.88% |
| C | 3.4151 | 4.1944 | 3.4765 | 0.7793 | 0.0614 | 7.88% |
| D | 3.9365 | 4.7240 | 3.9989 | 0.7875 | 0.0624 | 7.92% |
| E | 3.9921 | 4.7763 | 4.0544 | 0.7842 | 0.0623 | 7.94% |

| | |
|---|---|
| Aver- | 7.9% |
| age | |

The prepared silk fibroin solution with a concentration of 7.9% was used as a stock solution and diluted when used. The silk was purchased from Xinsilu Silk Sericulture Co., Ltd., Nantong City, Jiangsu Province. Anhydrous sodium carbonate, LiBr and PEG20000 were purchased from Sinopharm.

### Example 2: Solid-Phase Synthesis of Phosphorylated Micromolecular Polypeptide NYp

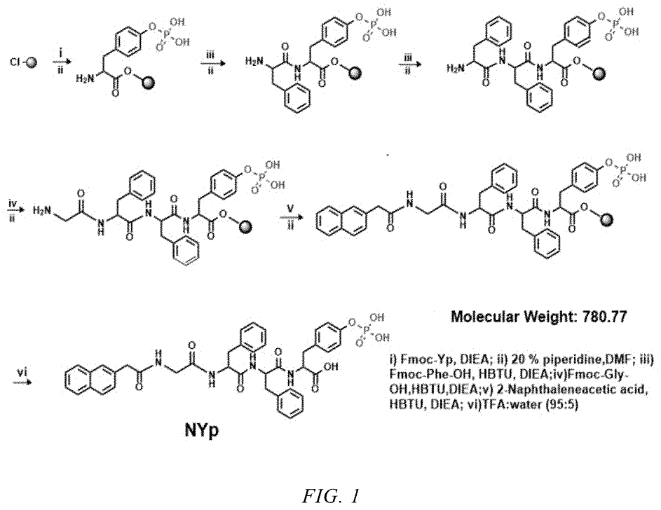

The synthetic process of a polypeptide molecule NYp was shown in FIG. 1. According to the sequence of the designed target molecule, using solid-phase synthesis technology, phosphotyrosine (Fmoc-Tyr(H₂PO₃)—OH), phenylalanine (Fmoc-Phe-OH), phenylalanine (Fmoc-Phe-OH), glycine (Fmoc-Gly-OH) and dinaphthylacetic acid (Nap) were added sequentially, and specific synthesis steps were as follows:

(1) Resin Swelling:

0.5 g of 2-chlorotrityl chloride resin was weighed and added into a solid phase synthesis reactor. Under the action of nitrogen, an appropriate amount of anhydrous dichloromethane (DCM) was added to swell the resin for 30 min, and then anhydrous DCM was squeezed out and the resin was washed 3 times with anhydrous N,N-dimethylformamide (DMF).

(2) Attachment to Fmoc-Tyr(H₂PO₃)—OH

0.845 g of Fmoc-Tyr(H₂PO₃)—OH was weighed and dissolved in 8 mL of anhydrous DMF and then 0.76 mL of DIEA was added thereto, ultrasound assisted dissolution was performed for complete dissolution, the resulting solution was added to the reactor, and the reaction proceeded for 1.5 h under nitrogen flow; then the reaction liquid was squeezed out and the resin was washed 4 times with anhydrous DMF.

(3) Resin Blocking

A blocking solution (DCM:MeOH:DIEA=80:15:5) was added into the reactor to react for 10 min under nitrogen flow, and the blocking reaction liquid was squeezed out; subsequently, the blocking solution was added again to react for 10 min, the blocking reaction liquid was squeezed out, and finally the resin was washed 4 times with anhydrous DMF.

(4) Deprotection of Fmoc Group

A formulated 20% piperidine solution (piperidine:DMF=20:80) was added into the reactor to react for 30 min under nitrogen flow, and then the resin was washed 3 times with the 20% piperidine solution and 4 times with anhydrous DMF, respectively.

(5) Attachment to Fmoc-Phe-OH

0.678 g of Fmoc-Phe-OH and 0.657 g of HBTU were weighed and dissolved in 8 mL of anhydrous DMF and then 0.76 mL of DIEA was added thereto, ultrasound assisted dissolution was performed for complete dissolution, the resulting solution was added to the reactor to react for 1 h under nitrogen flow; then the reaction liquid was squeezed out and the resin was washed 4 times with anhydrous DMF.

(6) Deprotection of Fmoc Group

A formulated 20% piperidine solution was added into the reactor, the reaction proceeded for 30 min under nitrogen flow, and then the resin was washed 3 times with the 20% piperidine solution and 4 times with anhydrous DMF, respectively.

(7) Attachment to Fmoc-Phe-OH

0.678 g of Fmoc-Phe-OH and 0.657 g of HBTU were weighed and dissolved in 8 mL of anhydrous DMF and then 0.76 mL of DIEA was added thereto, ultrasound assisted dissolution was performed for complete dissolution, the resulting solution was added to the reactor, to react for 1 h under nitrogen flow; then the reaction liquid was squeezed out and the resin was washed 4 times with anhydrous DMF.

(8) Deprotection of Fmoc Group

A formulated 20% piperidine solution was added into the reactor, the reaction proceeded for 30 min under nitrogen flow, and then the resin was washed 3 times with the 20% piperidine solution and 4 times with anhydrous DMF, respectively.

(9) Attachment to Fmoc-Gly-OH

0.52 g of Fmoc-Gly-OH and 0.657 g of HBTU were weighed and dissolved in 8 mL of anhydrous DMF and then 0.76 mL of DIEA was added thereto, ultrasound assisted dissolution was performed for complete dissolution, the resulting solution was added to the reactor to react for 1 h under nitrogen flow; then the reaction liquid was squeezed out and the resin was washed 4 times with anhydrous DMF.

(10) Deprotection of Fmoc Group

A formulated 20% piperidine solution was added into the reactor, the reaction proceeded for 30 min under nitrogen flow, and then the resin was washed 3 times with the 20% piperidine solution and 4 times with anhydrous DMF, respectively.

(11) Attachment to Nap

0.326 g of Nap and 0.657 g of HBTU were weighed and dissolved in 8 mL of anhydrous DMF and then 0.76 mL of DIEA was added thereto, ultrasound assisted dissolution was performed for complete dissolution, the resulting solution was added to the reactor to react for 1 h under nitrogen flow; then the reaction liquid was squeezed out and the resin was washed 4 times with anhydrous DMF.

(12) Resin Washing

The resin was washed 5 times with anhydrous DCM, anhydrous MeOH, and anhydrous n-hexane sequentially and then was blown dry with nitrogen.

(13) Polypeptide Separation

A 95% TFA solution (TFA:H₂O=95:5) was added into the reactor, the reaction proceeded for 2 h under nitrogen flow, then the reaction liquid was collected and the resin was washed 3 times with the 95% TFA solution, and then an air pump was used to blow away the TFA and a target product was precipitated with glacial diethyl ether. Finally, suction filtration was carried out to obtain the target product.

(14) Product Purification

Analytical and semi-preparative high performance liquid chromatography (HPLC) was used for separation and purification (water:acetonitrile=80:20 to 0:100), and freeze-drying treatment gave white powder NYp.

### Example 3

(1) 10 mg of NYp was weighed and dissolved in a glass vial, an appropriate amount of 1 mol/L NaOH was added to adjust the pH, and NYp was completely dissolved in ultrapure water to form a clear and transparent solution; then an appropriate amount of 1 mol/L HCl was added to make the pH of the system around 7.4, and deionized water was replenished to make up to a total volume of 2 mL, to obtain a NYp stock solution with a concentration of 0.5 wt %.

(2) A certain amount of the silk fibroin solution with a concentration of 7.9% was added to a glass vial, 1 mol/L NaOH was added to adjust the pH to about 7.4, and ultrapure water was added to volume, to obtain an SF stock solution with a concentration of 6.0% and pH=7.4.

(4) 5 μL of the SF stock solution was pipetted into a glass vial, then 30, 48, 60, 120 and 180 μL of the NYp stock solution were pipetted into the SF solution, respectively, and then 3 μL ALP was added, and finally the volume was made up to 300 μL, to obtain a mixed solution with an SF concentration of 0.1% and a NYp concentration of 0.05, 0.08, 0.1, 0.2 and 0.3 wt %, respectively.

(5) At room temperature, the glass vial was placed horizontally, and the gelation process was observed by inclination and inversion and recorded.

(6) In the same way, a mixed solution with a NYp concentration of 0.3 wt % and an SF concentration of 0.1, 0.5, 1.0, and 2.0% respectively could be prepared, and at room temperature, the gel state was observed and the time was recorded.

(7) In addition, a mixed solution with a NYp concentration of 0.3 wt %, an SF concentration of 2.0% and an ALP concentration of 10, 20 and 40 U/mL respectively could also be obtained, and at room temperature, the gel state was observed and the time was recorded.

Experimental Conditions of Rheological Test:

300 μL of a hydrogel sample was placed on 20 mm parallel plates and the rheological and mechanical test was conducted on the HAAKE RheoStress 600 rheometer produced by Thermo Scientific. The rotor type used in the test was PP20H, the working clearance of plates was 0.3 mm, the temperature was 25° C., and the mode was Controlled Deformation (CD). Strain sweep parameters: the frequency was 1.0 Hz, the strain sweep range was from 0.01% to 100%, and the step was 30. Frequency sweep parameters: the strain was 1.0%, the frequency sweep range was 0.1 Hz to 100 Hz, and the Decade was 9.

The properties of the gels formed by co-self-assembly of NYp and SF solutions of different concentrations under the catalysis of ALP are shown in Table 2:

TABLE 2

| Sample | Sol | Gel 1 | Gel 2 | Gel 3 | Gel 4 | Gel 5 | Gel 6 | Gel 7 | Gel 8 | Gel 9 |
|---|---|---|---|---|---|---|---|---|---|---|
| NapGFFYp (wt %) | 0.05 | 0.08 | 0.1 | 0.2 | 0.3 | 0.3 | 0.3 | 0.3 | 0.3 | 0.3 |
| SF (%) | 0.1 | 0.1 | 0.1 | 0.1 | 0.1 | 0.5 | 1.0 | 2.0 | 2.0 | 2.0 |
| pH | 7.4 | 7.4 | 7.4 | 7.4 | 7.4 | 7.4 | 7.4 | 7.4 | 7.4 | 7.4 |
| ALP (U/mL) | 10 | 10 | 10 | 10 | 10 | 10 | 10 | 10 | 20 | 40 |
| Gelation time (h) | — | 15 | 10 | 1 | 0.2 | 0.5 | 1 | 4 | 3 | 1.5 |
| G′ (Pa) | —^{a} | 27 | 37 | 83 | 165 | 607 | 1582 | 4865 | 5289 | 6147 |

^{a}The gelation process did not occur in 48 h.

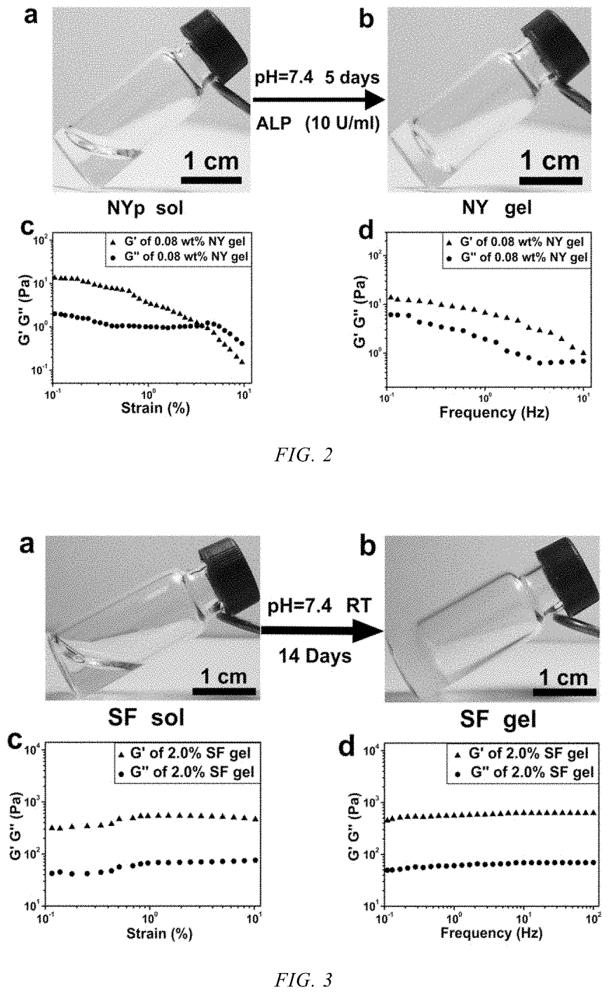

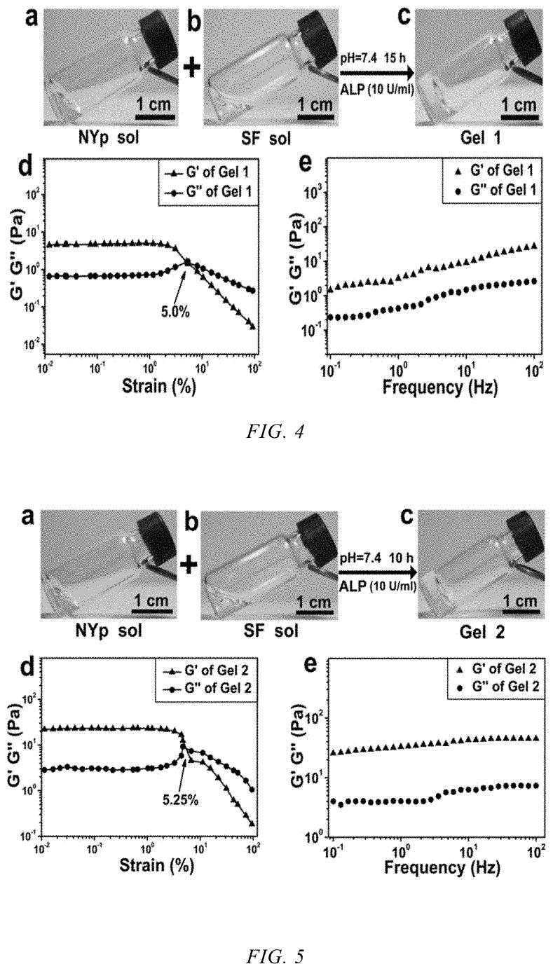

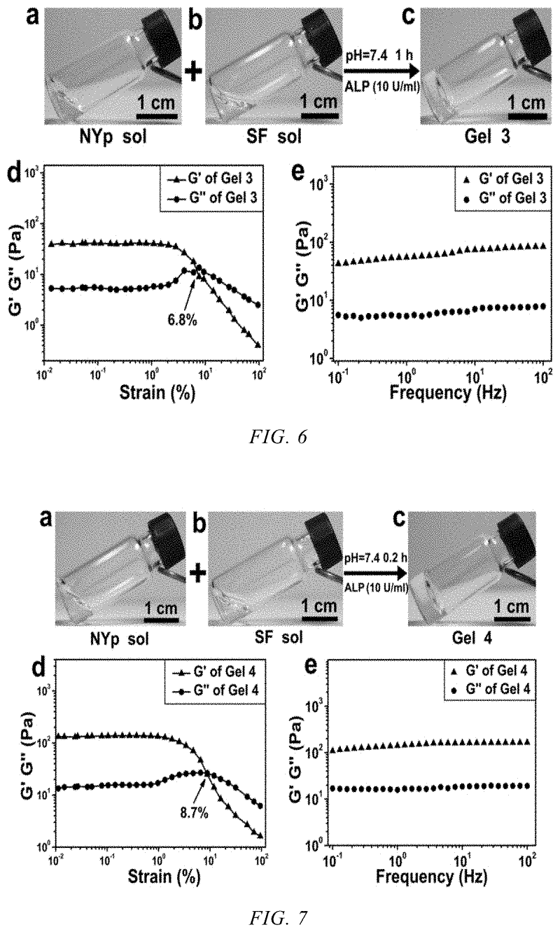

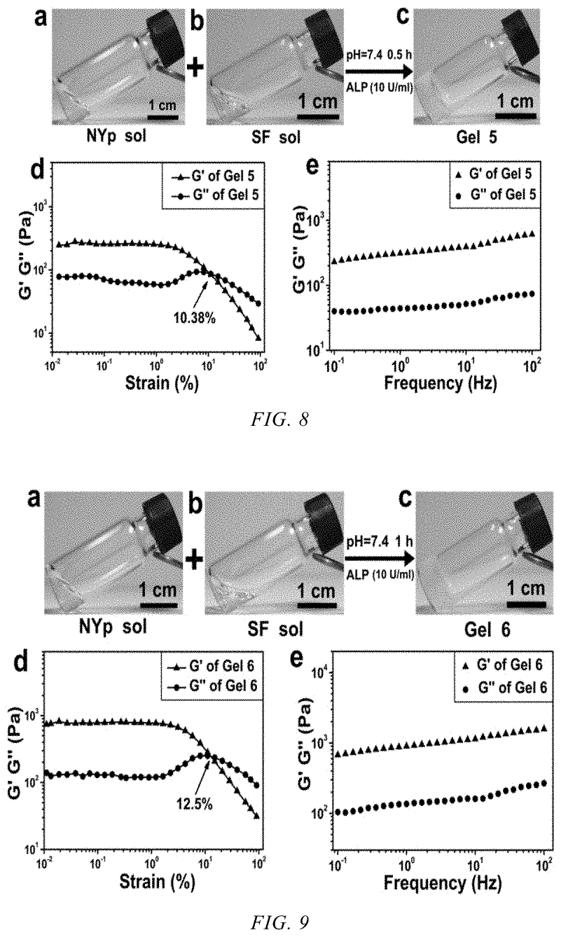

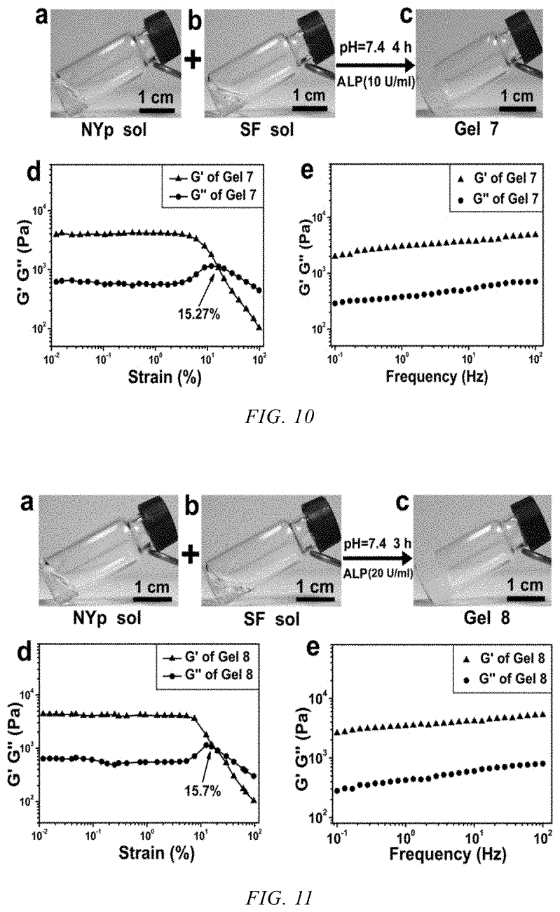

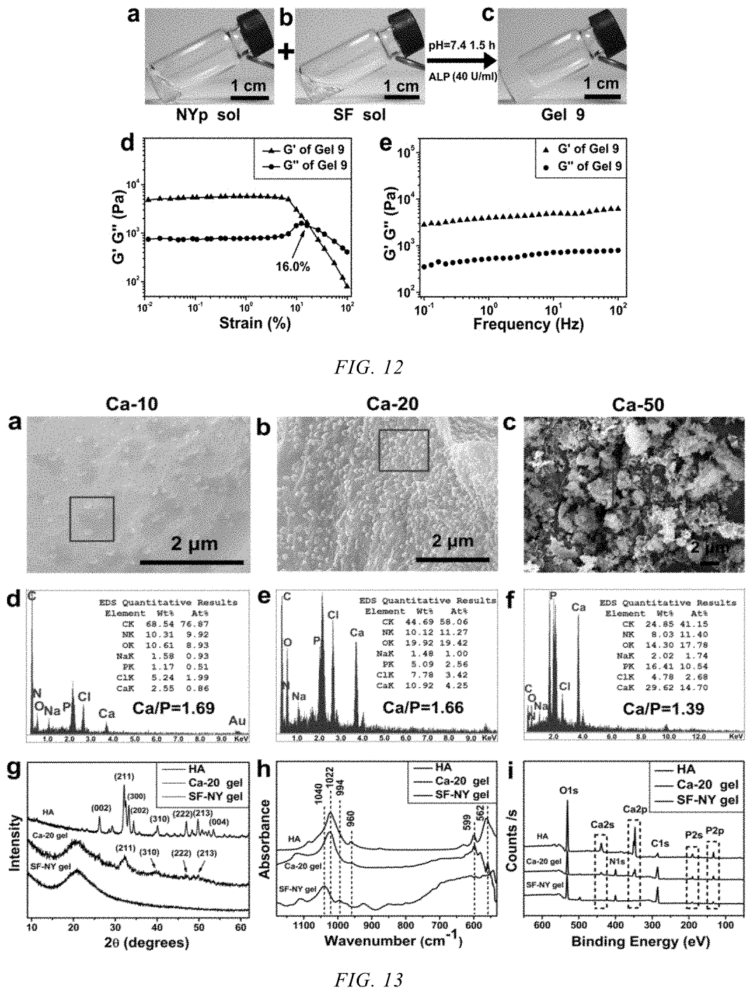

The characterization results of the gelation process and mechanical property are shown in FIGS. 2-12. At room temperature and under physiological conditions (pH=7.4), it takes at least 14 days for an SF solution with a concentration of 2.0% to form a hydrogel (FIG. 3). The SF concentration is fixed at 0.1%, the ALP concentration is fixed at 10 U/mL, and the NYp concentration is 0.05, 0.08, 0.1, 0.2, and 0.3 wt % respectively. The experimental research results show that when the NYp concentration is 0.05 wt %, the 0.1% SF solution cannot be induced to form a hydrogel. When the NYp concentration increases from 0.08 wt % to 0.3 wt %, the gelation time is reduced from 15 h to 0.2 h, and the storage modulus (G′) of the gel increases from 27 Pa to 165 Pa (FIGS. 4-7). Similarly, when the NYp concentration is fixed at 0.3 wt %, the ALP concentration is fixed at 10 U/mL, and the concentration of the SF solution is 0.1%, 0.5%, 1.0% and 2.0% respectively, the gelation time increases from 0.2 h to 0.5 h, 1 h and 4 h; and also the storage modulus (G′) of the gel increases from 165 Pa to 607 Pa, 1582 Pa and 4865 Pa (FIGS. 7-10). In addition, the ALP concentration can also be increased to reduce the gelation time and increase the mechanical property. For example, when the NYp concentration is fixed at 0.3 wt %, the SF concentration is fixed at 2.0%, and the ALP concentration increases from 10 U/mL to 20 U/mL and 40 U/mL, the gelation time decreases from 4 h to 3 h and 1.5 h respectively, and also the storage modulus (G′) of the gel increases from 4865 Pa to 5289 Pa and 6147 Pa, respectively (FIGS. 10-12). These experimental research results show that NYp is an excellent gelator precursor, which can be catalyzed by ALP to trigger supramolecular self-assembly, forming a NY supramolecular hydrogel, and to synergistically induce silk fibroin co-self-assembly, forming a stable SF hydrogel.

### Example 4: Preparation and Characterization of a Biomimetic Mineralized Hydrogel

Preparation of different concentrations of a mineralizing liquid: (a) CaCl₂=10 mM, β-GP=6 mM; (b) CaCl₂=20 mM, β-GP=12 mM; and (c) CaCl₂=50 mM, β-GP=30 mM; once the SF-NY hydrogel (NY=0.3 wt %, SF=2.0%, ALP=10 U/mL) was stable, the mineralizing liquids a, b, and c were respectively added for 7 days of culture, and the products were denoted as Ca-10 gel, Ca-20 gel and Ca-50 gel, respectively.

The results are shown in FIG. 13. When Ca²⁺=10 mM, a small amount of microcrystals appear on the pore wall of the SF-NY gel material (FIG. 13a). When Ca⁺²=20 mM, a large number of microspherical crystals are uniformly distributed on the pore wall of the SF-NY hydrogel material (FIG. 13b). However, when the Ca²⁺ concentration is further increased (Ca²⁺=50 mM), flower-like aggregates are observed in the SF hydrogel (FIG. 13c). EDS analysis results show that when Ca²⁺=10 mM and Ca²⁺=20 mM, the atomic ratios of calcium to phosphorus are 1.69 and 1.66, respectively, which are close to the calcium to phosphorus ratio 1.67 of hydroxyapatite, the main inorganic component of natural bone. However, when Ca²⁺=50 mM, the atomic ratio of calcium to phosphorus is 1.39, which is lower than the calcium to phosphorus ratio of hydroxyapatite of 1.67. These experimental results show that an appropriate amount of Ca²⁺ concentration is essential to regulate the nucleation and growth of calcium phosphate crystals in SF-NY hydrogels. In order to study the crystal phase composition of the biomimetic mineralized Ca-20 hydrogel, X-ray diffraction (XRD) test was conducted, and the results are shown in FIG. 13g. The broad diffraction peaks of SF-NY hydrogel and Ca-20 hydrogel at 2θ=20.5° are characteristic diffraction peaks of silk fibroin, indicating that the secondary structure of silk fibroin is a stable β-sheet structure. For Ca-20 hydrogel, in addition to one broad diffraction peak at 20.5°, 4 diffraction peaks also appear at 31.9°, 40°, 46.9° and 49.8°, which are attributed to (211), (310), (222) and (213) of hydroxyapatite (HA), respectively. In addition, Fourier transform infrared spectroscopy (FTIR) and X-ray photoelectron spectroscopy (XPS) tests were used to further study the structural information of the mineral phase in the Ca-20 hydrogel. It can be clearly seen from FIG. 13h that the infrared spectra of the hydrogel before and after biomimetic mineralization have obvious differences. Compared with the SF-NY hydrogel, the infrared spectrum of the biomimetic mineralized Ca-20 hydrogel shows four new absorption peaks at 1022 cm⁻¹, 960 cm⁻¹, 599 cm⁻¹ and 562 cm⁻¹, which are mainly due to the molecular vibration of the phosphate group. As shown in FIG. 13i, for the SF-NY hydrogel and the Ca-20 hydrogel, three peaks appear at 285 eV, 400 eV and 532 eV, respectively, which are attributed to C 1s, N 1s and O 1s, respectively. In addition, in the XPS spectrum of the Ca-20 hydrogel, four peaks appear at 134 eV, 190 eV, 347 eV, and 439 eV, respectively, which are attributed to P 2p, P 2s, Ca 2p, and Ca 2s, respectively.

### Example 5: Biocompatibility Evaluation

The Live/Dead staining method was used to evaluate the cell biocompatibility of a mixed hydrogel (SF-NY gel) and a biomimetic mineralized hydrogel (Ca-20 gel) with rat bone marrow mesenchymal stem cells (rBMSCs). 80 μL of SF-NY gel and Ca-20 gel were placed in an 8-well glass confocal plate (Nunc 155409), and then rBMSC cells were inoculated on the surface of the gel at a cell density of 1.5×10⁴/cm², and then they were cultured in a 37° C., 5% CO₂ cell incubator, and the medium was refreshed every other day. After 1, 4, and 7 days of culture, the cells were stained with calcein-AM/propidium iodide-PI, and the morphology of rBMSC cells on the gel surface was observed under a fluorescence microscope (Olympus IX71, Olympus) and photographed and recorded; the cell density was calculated using Image J software. The CCK8 method was used to further evaluate the cell viability and proliferation of rat bone marrow mesenchymal stem cells (rBMSCs) on the surface of SF-NY gel and Ca-20 gel, respectively. After 1, 4, and 7 days of culture, a culture containing a 10% CCK-8 solution was added to a 12-well plate. After culturing in a cell incubator for 2 h, 100 μL of the mixed culture was drawn from each well and added into a new 96-well plate. The 96-well plate was put into a multi-mode microplate reader of Thermo Fisher Scientific and the optical density value (OD value) of each well was recorded at a wavelength of 450 nm.

The results are shown in FIG. 14. From the green fluorescence of the cells and the polyhedral morphology of most cells, it can be seen that the inoculated rBMSCs can adhere well to the surface of SF-NY hydrogel and Ca-20 hydrogel, and show good cell viability after 1 day of culture. In addition, with the increase of the culture time, rBMSCs can grow rapidly and proliferate well on the surface of the hydrogel. For example, the cell density of rBMSCs on the surface of Ca-20 hydrogel increases from 1.852×10⁴ cm⁻² on day 1 to 7.1067×10⁴ cm⁻² on day 7, which is slightly higher than those of the corresponding SF-NY hydrogel and the blank control (FIG. 14b). The CCK8 method further confirms the high cell viability of rBMSCs cultured on the surface of Ca-20 hydrogel (FIG. 14c). These experimental results show that SF-NY hydrogel and Ca-20 hydrogel have good biocompatibility, and when used as cell culture scaffold materials, they are beneficial to the adhesion, growth and proliferation of rBMSCs on their surface.

### Example 6: Evaluation of Osteogenic Differentiation In Vitro

In order to further evaluate the osteogenic induction ability of the biomimetic mineralized Ca-20 gel hydrogel on rat bone marrow mesenchymal stem cells (rBMSCs), real-time quantitative reverse transcription polymerase chain reaction (qRT-PCR) was used to detect the expression of osteogenesis-related genes and proteins, including transcription factor (Runx2), type I collagen (Col1a), osteocalcin (OCN) and osteopontin (OPN), etc. 300 μL of Ca-20 hydrogel was plated onto a 12-well plate, and then immersed in fresh DMEM and incubated for 30 min. After that, the medium was removed, and the cells were inoculated on the surface of Ca-20 hydrogel at a density of 1×10⁵ cells per well to grow on the surface of the Ca-20 hydrogel in a normal growth medium. After 24 h of culture, the normal medium was replaced with osteoinductive medium. At different time points (4, 7 and 14 days), total RNA was extracted using TRIzol kit (Invitrogen, USA). Then, 1 μg of total RNA was reverse transcribed using PrimeScript RT kit (TakaRa, Japan) according to the manufacturer's instructions, to obtain complementary DNA. Then, qRT-PCR was performed using SYBR Green qRT-PCR kit (TakaRa, Japan) and ABI Step One Plus real-time PCR system (Applied Biosystems, USA). The experimental data was processed by the 2-ΔΔCt method. GAPDH was used as a reference, and each sample was repeated three times. Cells cultured on SF-NY gel under the same conditions and a blank culture plate served as control.

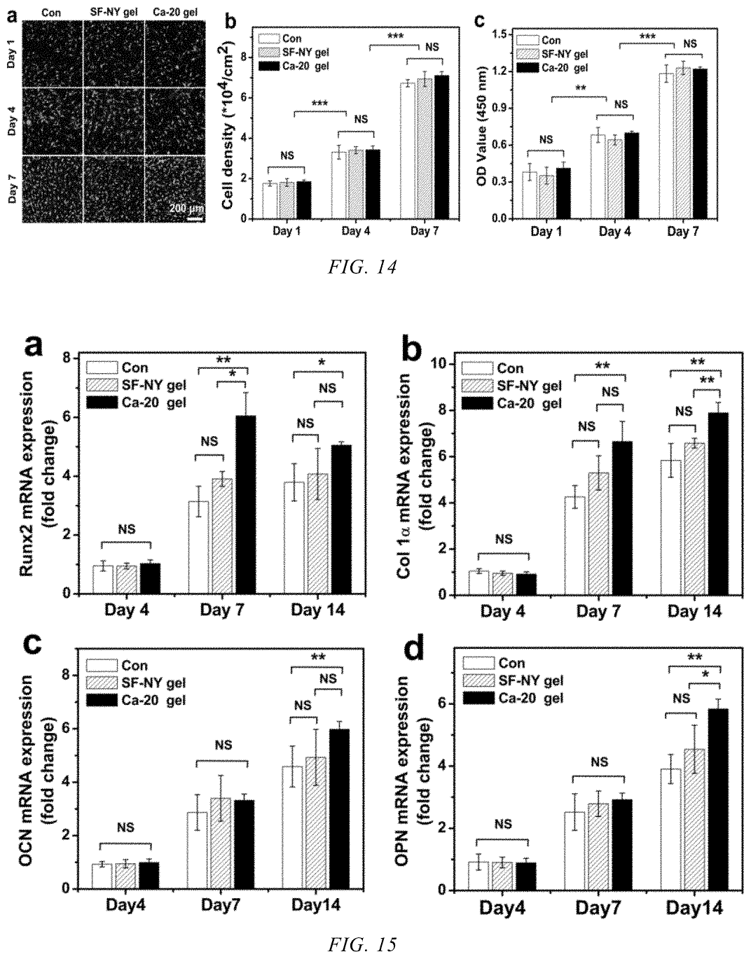

The results are shown in FIG. 15. After 1 day of culture, there is no significant difference in the expression of osteogenic differentiation-related markers such as Runx2, Col1α, OCN, OPN in Ca-20 hydrogel, compared with SF-NY hydrogel and the blank control. However, after 7 days and 14 days of culture, the expression of all osteogenic differentiation-related markers, including Runx2, Col1α, OCN and OPN, is significantly up-regulated. In addition, after 7 days and 14 days of culture, the gene expression levels of rBMSCs cultured on the surface of Ca-20 hydrogel are higher than those of the corresponding SF-NY hydrogel group and the blank control group. These experimental results show that, compared with SF-NY hydrogel and the blank control, Ca-20 hydrogel has a better ability to promote osteogenic differentiation.

### Example 7: Evaluation of Bone Regeneration Ability In Vivo

After 4 and 8 weeks of postoperative observation, the femur was removed and fixed with 10% formalin. A micro CT machine (mCT-80, Scanco Medical, Bassersdorf, Switzerland) was used to evaluate the morphology of the femur. The CT parameters were set as follows, pixel matrix: 1024×1024; resolution: 20 μm. Scanco software was used to perform 3D reconstruction analysis on the scanned images, and bone mineral density (BMD), bone volume to total tissue volume ratio (BV/TV), trabecular thickness (Tb.Th), and trabecular space (Tb.Sp) were analyzed.

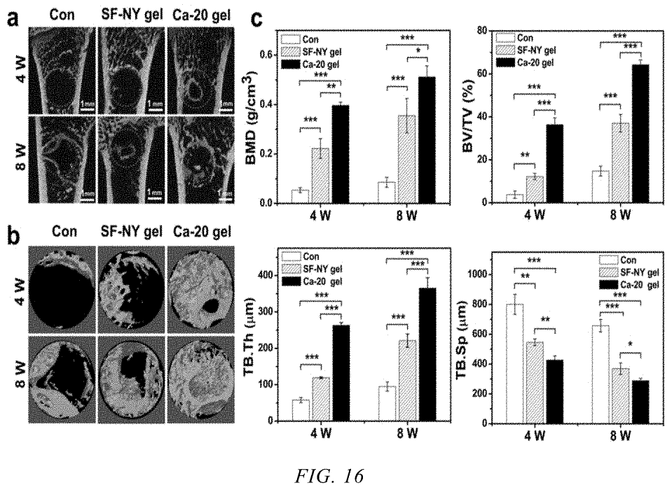

The results are shown in FIG. 16. After 4 weeks, more new bone tissue was formed in the femoral defect site where the Ca-20 hydrogel is implanted, compared with the SF-NY hydrogel group and the blank control group. Moreover, with the increase of time, more new bone tissue is formed in the bone defect site. In addition, further quantitative analysis of the newly formed bone tissue was performed by Micro-CT: bone mineral density (BMD), bone volume to total tissue volume ratio (BV/TV), trabecular thickness (Tb.Th), and trabecular space (Tb.Sp). These factors are important evaluation indicators of bone regeneration ability. As shown in FIG. 16c, at 4 weeks and 8 weeks, respectively, compared with the SF-NY hydrogel group and the blank control group, the Ca-20 hydrogel group has the highest BMD, BV/TV and Tb.Th and the lowest Tb.Sp, which shows that Ca-20 hydrogel can significantly promote the regeneration of bone tissue in the femoral defect site of rats.

The examples described above are only preferred examples for fully illustrating the present invention, and the protection scope of the present invention is not limited thereto. Equivalent substitutions or changes made by those skilled in the art on the basis of the present invention are all within the protection scope of the present invention. The protection scope of the present invention is defined by the claims.

1. A method for inducing gelation of a silk fibroin solution by alkaline phosphatase, comprising the following steps:
adding a self-assembling micromolecular polypeptide in a silk fibroin solution as a gelator precursor to obtain a mixed solution, and adding alkaline phosphatase into the mixed solution to remove a phosphate group on the molecule of the self-assembling micromolecular polypeptide, to trigger supramolecular self-assembly and induce silk fibroin co-self-assembly, forming a silk fibroin gel material.

2. The method according to claim 1, wherein the self-assembling micromolecular polypeptide is selected from 2-naphthalene acetic acid-glycine-phenylalanine-phenylalanine-phosphotyrosine, 2-naphthalene acetic acid-phenylalanine-phenylalanine-lysine-phosphotyrosine, 2-naphthalene acetic acid-phenylalanine-phenylalanine-phosphotyrosine and any combination thereof.

3. The method according to claim 1, wherein the concentration of the silk fibroin in the mixed solution is 0.1%-2.0%.

4. The method according to claim 1, wherein the concentration of the self-assembling micromolecular polypeptide in the mixed solution is 0.05 wt %-0.3 wt %.

5. The method according to claim 1, wherein the amount of the alkaline phosphatase added is 10 U/mL-40 U/mL.

6. The method according to claim 1, wherein the pH of the mixed solution is 7-8.

7. A silk fibroin gel material prepared by the method of claim 1.

8. A method for biomimetic mineralization of the silk fibroin gel material of claim 7, comprising adding the silk fibroin gel material into a mineralizing solution to culture for 5-10 days to obtain a biomimetic mineralized hydrogel, the mineralizing solution comprising 10-40 mM CaCl₂ and 6-20 mM β-glycerophosphate.

9. A biomimetic mineralized hydrogel prepared by the method of claim 8.

10. Use of the biomimetic mineralized hydrogel of claim 9 in the preparation of body tissue repair materials.
